(19) United States
(12) Patent Application Publication
(10) Pub. No.: US 2007/0064870 A1
(43) Pub. Date: Mar. 22, 2007

(54) Patient support method of examining a patient and examining facility

(76) Inventors: Klaus Herrmann, Nurnberg (DE); Eike Rietzel, Darmstadt (DE); Andres Sommer, Langensendelbach (DE)

Correspondence Address: BRINKS HOFER GILSON & LIONE, P.O. BOX 10395, CHICAGO, IL, 60610, US

(21) Appl. No.: 11/520,269

(22) Filed: Sep. 13, 2006

Related U.S. Application Data
(60) Provisional application No. 60/717,832, filed on Sep. 16, 2005.

(30) Foreign Application Priority Data
Sep. 16, 2005 (DE) 10 2005 044 415.6

(51) Int. Cl. A61N 5/10

(52) U.S. Cl. 378/65

(57) ABSTRACT

A method for examining and treating a patient, patient room and examining device are provided. The method comprising positioning the patient on a first patient support for an examination, examining the patient with an imaging examining device, positioning the patient on a second patient support having a support surface identically to the first patient support, and irradiating the patient with an irradiating device.

The present patent document claims the benefit of the filing date under 35 U.S.C. §119(e) of Provisional U.S. Patent Application Ser. No. 60/717,832, filed on Sep. 16, 2005, which is hereby incorporated by reference. This application also claims the benefit of DE 10 2005 044 415.6, filed Sep. 16, 2005.

### BACKGROUND

1. Field

The present embodiments relate to a patient support method of examining and treating a patient and to an examining device and a patient room or examining facility.

2. Related Art

Generally, before a radiation therapy is carried out, the treatment region of a patient to be irradiated is normally examined with an imaging diagnostic device, for example, a computer tomography (CT) scanner. To obtain recordings with high resolution that are accurate, the patient is immobilized on a patient bed or support and the patient support is moved at least partially through the examining device. The recordings produced as a result are used for obtaining a data record. An irradiation plan is worked out in accordance with the data record. The patient is exposed to the radiation therapy in one or more sessions in accordance with the irradiation plan. The radiation therapy takes place in a room separate from the area where the irradiating device, for example, a linear accelerator, is installed. Before the radiation therapy, the patient is repositioned on a second patient support in accordance with the irradiating device. The patient should assume the same position with his body.

Obtaining a desired orientation of the second patient support with respect to the irradiating device and an identical body position of the patient on both patient supports is difficult, for example, when using a computer tomography scanner as an examining device. The circular aperture of the computer tomography scanner is kept as small as possible, so that the imaging unit of a computer tomography scanner can move as close as possible to the patient. Generally, a special shape of a conventional CT patient support is used. The special shape of the conventional CT patient support has less width than a patient support of an irradiating device, is relatively thin and has a hollow molding in which the patient is placed during the examination.

Conventionally, the hollow molding is filled with an insert having a convex side as disclosed, for example, in German document DE 201 21 050 U1. Radiation therapy planning takes place after the hollow molding is filled with the insert.

The conventional patient support of the computer tomography scanner, due to its lack of strength, bends by different amounts when moving the support into the computer tomography (CT) scanner, depending on the scanning position. The geometric distortion arises during the image recordings. If the intention is to irradiate by using the second patient support, the normal procedure is to eliminate the first patient support from the data record obtained by the CT recordings and to supplement it with the data record of the second patient support that is used during the radiation therapy.

### SUMMARY

One embodiment includes positioning the patient on a first patient support for an examination, examining the patient with an imaging or examining device, positioning the patient on a second patient support having a support surface identical to the first patient support, and irradiating the patient with an irradiating device.

In this embodiment, at least high precision may result while simultaneously simplifying the entire treatment process. In another embodiment, patient supports with identically constructed support surfaces for the examination and for the treatment are used. This may eliminate the correction for the patient support in the radiation therapy planning. The identical mechanical characteristics of identically constructed support surfaces, interfering bending effects arising from the shape and the structure of the patient support may also be omitted.

In one embodiment, the gray scale values measured with the imaging or examining device during the examination and the information about the geometric shape of the support surface are stored. The parameters related to the geometry of the patient support are the same. The information about the patient support obtained during the examination does not need to be deleted from the data record of the examination and replaced by other data. The information about the position of the patient and about the patient support can simply continue to be used during the irradiation, resulting in a more simple procedure. In one embodiment, using identically constructed support surfaces may avoid problems in the immobilization of the patient that arise due to the previously different support surfaces in the examination and the treatment.

In one embodiment, the patient is positioned for treatment on a patient support that has a positioning mechanism that is constructed identical to the first patient support. In one embodiment, the positioning mechanism is a base on which the patient support is supported. In another embodiment, the positioning mechanism comprises a number of moving components with the aid of which the patient support can be adjusted or moved.

In one embodiment, the same patient support is used for examination and for treatment. This may reduce cost and simplify the method of examination. In one embodiment, the patient support is not moved but the examining device and/or irradiating device are moved. The patient only needs to be immobilized once on the patient support for examination, and must neither be repositioned on another patient support nor brought to another location for the treatment.

In one embodiment, after the examination, the patient support is moved towards the irradiating device. For example, when the examining device and the irradiating device are not designed in such a manner that both of them can be moved over the same fixed patient support, after the examination, the patient support is moved towards the irradiating device. The patient does not have to be repositioned onto another support. The space coordinates of the patient support within a patient room are changed and the tissue to be treated is oriented towards the irradiating device in the same way as towards the examining device. In one embodiment, the patient support is adjusted in the vertical direction.

In one embodiment, the examination and the treatment of a patient take place in two separate rooms, which may be far away from one another. For example, the examining and treatment devices are constructed in such a manner that the associated patient supports are connected inseparably to the devices so that it is very difficult or even impossible to move the patient support from one device to the other. In one embodiment, the patient is positioned on the first patient support for examination and on a second separate patient support for treatment. The two separate patient supports have the same support surfaces so that equalizing may no longer be necessary. In an alternate embodiment, the same patient support is used for diagnosis and for radiation therapy.

In one embodiment, the first and the second patient supports are adjusted into identical positions for examination and for treatment using an identical positioning mechanism. Both patient supports experience the same adjustment because of the identical positioning mechanism. The support surface of a conventional patient support for a computer tomography scanner is, for example, very thin and bends under the weight of the patient. In one embodiment, the magnitude of bending during examination and during treatment is identical and when the two identical patient supports are adjusted.

In one embodiment, the spatial position of the first patient support is detected by a computer unit in a data record and this data record is used for an automated adjustment of the second patient support using its positioning mechanism. The two patient supports are brought into the same position with very high accuracy.

In one embodiment, the treatment with the irradiating device is performed in a different room from the examination with the examining device. In this embodiment, the two rooms may be spatially far apart, for example, when the two rooms are in different buildings or even in different geographic locations.

In one embodiment, the support surface of the patient support is included in an irradiation plan during the irradiation of the tissue to be treated. The data about the first patient support are no longer replaced. Formulating the irradiation plan may become much quicker and simpler, particularly if it is intended to radiate through the support surface.

In one embodiment, a patient room is an examining place at which an examining device, for example, a computer tomography scanner, is installed. In another embodiment, the patient room is a treatment place at which an irradiating device is installed. Patient supports with identically constructed support surfaces can be positioned at the examining device and at the irradiating device. A patient room may be, for example, a large room, a hospital department, a doctor's practice, a clinic or another facility with a diagnostic and/or treatment place per se or in combination.

In another embodiment, an examining device for performing a medical examination of a patient is a positionable patient support. The support surface of which is constructed for positioning at an irradiating device.

In one embodiment, the same patient support can be used both at the examining device and at the treatment device. The patient support is both movable and immobile. In both cases, for example, the patient support is separable from the examining device. A variant of the examining device provides for the moving patient support to be simply moved to the treatment device after the examination. According to another variant, the patient support is immobile and the examining device is moved over the patient support for diagnosis and is then moved away. The irradiating device can also be adjustable even when it is mounted in one place on a wall or on a ceiling of the room.

### BRIEF DESCRIPTION OF THE DRAWINGS

FIG. 1 shows a patient support according to one embodiment with a patient positioned thereon during a computer tomography scanner examination,

FIG. 2 shows a patient support according to one embodiment with a patient positioned thereon during an examination with a further imaging device, and

FIG. 3 shows a patient support according to one embodiment with a patient positioned thereon during an irradiation with a fixed-beam irradiating device.

### DETAILED DESCRIPTION

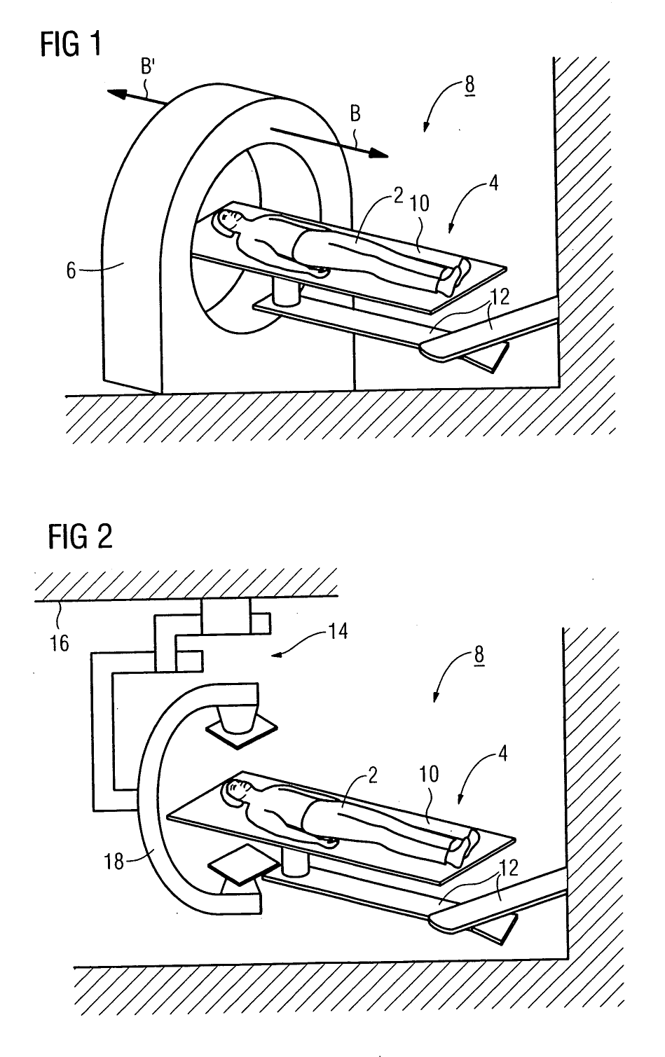

In one embodiment, as shown in FIG. 1, a patient 2 is positioned for examination on a patient support 4 and is immobilized. The examination is performed with an imaging or examining device 6, for example, a computer tomography scanner. The patient support 4 and the computer tomography scanner 6 are located in a patient room 8.

The patient support 4 includes a support surface 10 and a positioning mechanism 12 that are movable into a specific position. The positioning mechanism 12 may include many different components and constructed with different complexities. In one embodiment, when the patient support 4 is constructed to be immobile, the positioning mechanism 12 comprises, for example, a stationary base. In another embodiment, the positioning mechanism 12 has a number of components that are movably connected to one another and allow the patient support 4 to be adjusted in one, more or all directions and/or moved to another location.

In one embodiment, the support surface 10 includes an area of the patient support 4 on which the patient 2 is supported and is constructed in the manner of a thin plate.

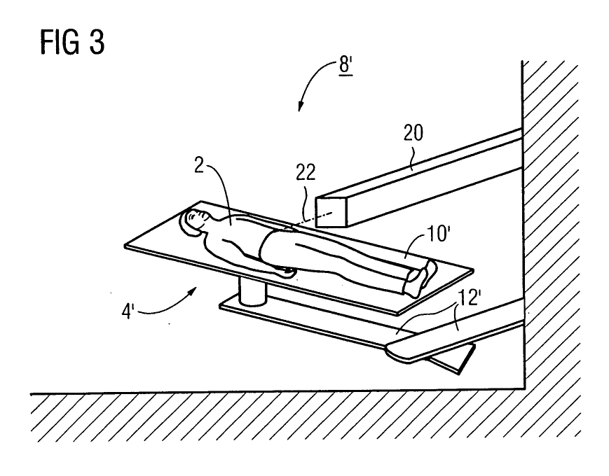

In another embodiment, the patient support 4, including the support surface 10 and the positioning mechanism 12, also has sensors, not shown, which detect the spatial position of the support surface 10 and/or of the positioning mechanism 12. This data is sent to a computer unit so that the information can be stored in a data record. This data record is used later for automatically adjusting a patient support 4′, as shown in FIG. 3, on which the patient 2 is positioned for treatment, into exactly the same position.

In one embodiment, the computer tomography scanner 6 includes a circular aperture that is movable around the support surface 10 of the patient support 4, which is indicated by arrows B and B′. The computer tomography scanner 6 is used for making recordings of regions on or in the body of the patient 2 and of the support surface 10. During the examination, information about the geometry, dimensions and orientation of the patient support 4 is obtained and stored in the data record and at least a part of this information is obtained from the gray scale values of the CT recordings. This information, together with the data about the exact positioning of the patient 2, is used in the radiation therapy planning.

In another embodiment, as shown in FIG. 2, the patient support 4 is operable with an alternative imaging device 14. In one embodiment, in contrast to the computer tomography scanner 6, the examining device 14 is mounted on the ceiling 16 of the room 8. The examining device 14 has a multi-component structure by means of which a C-shaped examining unit 18 is adjusted in all directions in order to create recordings of a tissue to be treated.

In another embodiment, as shown in FIG. 3, a patient support 4′ supports a patient 2 during a treatment in a patient room 8′. The patient support 4′, the support surface 10′ and the positioning mechanism 12′ have an identical structure to the respective elements according to FIG. 1 and FIG. 2. In one embodiment, the patient supports 4 and 4′ are the same patient support. The patient rooms 8 and 8′ can be one and the same, for example, if the patient supports 4 and 4′ are stationary and has a restricted freedom of movement. In an alternate embodiment, the two patient rooms 8 and 8′ are two separate rooms, which have a great spatial distance from one another, for example, two rooms in two different departments of a hospital.

In one exemplary embodiment, as shown in FIG. 3, an irradiating device 20 is a fixed-beam device that is mounted on a wall of the examining room 8′. A beam 22 is directed onto the desired (diseased) tissue of the patient 2 through a beam outlet of the irradiating device 20. For example, when a fixed-beam device 20 is used, the direction of the beam 22 is fixed and only the patient support 4 is adjusted in order to bring the patient 2 into precisely the position in which his desired (diseased) tissue is treated. In other embodiments, alternative irradiating devices 20 are used and the mobility is less restricted, for example, the patient 2 can be irradiated from different directions by adjusting the irradiating device 20.

In one embodiment, the data record obtained during the examination is used to adjust the irradiating device 20 and the patient support 4′ with respect to one another via a control unit. If the patient support 4′ is a second separate patient support 4′, it is adjusted into the position required for the irradiation by the positioning mechanism 12′.

In another embodiment, the stored information about the support surface 10 is also used to generate an irradiation plan. Since the two patient supports 4 and 4′ are identical or even the same, the support surfaces 10 and 10′ do not need to be eliminated in the radiation therapy planning. In this embodiment, the data obtained by recordings generated by an imaging method continue to be used and may simplify the treatment.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

1. A method for examining and treating a patient, the method comprising:
positioning the patient on a first patient support for an examination,
examining the patient with an imaging device,
positioning the patient on a second patient support having a support surface identical to the first patient support, and
irradiating the patient using a treatment irradiating device.

2. The method as claimed in claim 1, wherein the imaging device is a computer tomography scanner.

3. The method as claimed in claim 2, wherein a tissue of the patient is irradiated.

4. The method as claimed in claim 1, wherein positioning the patient for treatment on the second patient support comprises positioning with a second positioning mechanism identical to a first positioning mechanism of the first patient support.

5. The method as claimed in claim 1, further comprising: using the first patient support as the second patient support, the same patient support being used when examining the patient and irradiating the patient.

6. The method as claimed in claim 5, further comprising: moving the first patient support towards the irradiating device after examining the patient.

7. The method as claimed in claim 1, further comprising: positioning the patient on the first patient support when examining the patient and on the second patient support when irradiating the patient, the first patient support separate from the second patient support.

8. The method as claimed in claim 7, further comprising: adjusting the first and second patient support into identical positions when examining and irradiating the patient using identical positioning mechanisms.

9. The method as claimed in claim 8, further comprising: detecting a spatial position of the first patient support by a computer unit for a data record and automatically adjusting the second patient support using a positioning mechanism and the data record.

10. The method as claimed in claim 1, wherein examining the patient and irradiating the patient are performed in different rooms.

11. The method as claimed in claim 1, further comprising: including a support surface of the first patient support in an irradiation plan during the irradiation of the patient.

12. A patient room comprising an examining device, and an irradiating device, wherein a first patient support and second patient support comprise identical support surfaces and are operable to be positioned at the examining device and at the irradiating device.

13. The patient room as claimed in claim 12, wherein the examining device is a computer tomography scanner.

14. The patient room as claimed in claim 12, wherein the first patient support has a positioning mechanism support that can be moved from the examining device to the treatment device.

15. An examining device for performing a medical examination of a patient, the examining device comprising a positionable patient support, a support surface of the patient support being constructed for positioning at an irradiating device.

16. The examining device as claimed in claim 15, wherein the patient support is positioned at the examining device.
